(19) United States
(12) Patent Application Publication
(10) Pub. No.: US 2026/0010761 A1
(43) Pub. Date: Jan. 8, 2026

(54) SYSTEM AND METHOD FOR PREDICTIVE ANALYSIS OF 2-DIMENSIONAL CRYSTAL STRUCTURES

(71) Applicant: Constructor Technology AG, Schaffhausen (CH)

(72) Inventors: Andrey Ustyuzhanin, Singapore (SG); Egor Shibaev, Singapore (SG); Laurent Dedenis, Geneve (CH); Stanislav Protasov, Singapore (SG); Serg Bell, Singapore (SG); Nikolay Dobrovolskiy, Alanya (TR)

(73) Assignee: Constructor Technology AG, Schaffhausen (CH)

(21) Appl. No.: 19/255,999

(22) Filed: Jun. 30, 2025

Related U.S. Application Data
(60) Provisional application No. 63/667,107, filed on Jul. 2, 2024.

(51) Int. Cl. G06N 3/045; G06N 3/0464; G06N 3/088; G16C 20/30

(52) U.S. Cl. CPC G06N 3/045; G06N 3/0464; G06N 3/088; G16C 20/30

(57) ABSTRACT

The present invention provides a system and method for applying Siamese Neural Networks (“SNNs”) to model, characterize, and predict the effects of defects on material properties, specifically for 2-dimensional (“2D”) crystals such as transition metal dichalcogenides (“TMDCs”). The present invention provides a method for predicting physical properties with strong performance across both low and high-defect density scenarios.

### CROSS REFERENCE TO RELATED APPLICATIONS

This application claims the benefit of U.S. Provisional Patent Application Ser. No. 63/667,107, filed Jul. 2, 2024, the contents of which are incorporated herein by reference.

### BACKGROUND OF THE INVENTION

The present invention is directed to a novel method for performing predictive analysis of defect configurations in 2-dimensional (“2D”) crystal structures. 2D crystals, particularly transition metal dichalcogenides (“TMDCs”) such as molybdenum disulfide MoS₂, have received significant attention due to their unique electronic, optical, and mechanical properties which make them suitable for a wide range of applications, from transistors to photovoltaic devices, and further provide a fertile ground for research efforts toward comprehending and manipulating crystal defects. Furthermore, these materials feature a distinctive structure, comprising a plane of metal atoms sandwiched between planes of chalcogen atoms. This layered configuration is crucial in determining its physical and chemical properties.

The present invention provides a solution to an urgent need for computational tools that can efficiently and precisely model, characterize, and predict the effects of defects on material properties. Specifically, the present invention provides a capability to predict physical properties such as formation energy per site and the bandgap with strong performance across both low and high-defect density scenarios, outperforming previous traditional methods when enhanced with novel polynomial features. Furthermore, the present invention offers a robust method for efficient representation and retrieval of complex defect configurations, thereby facilitating faster and more accurate predictions of desired material properties.

The entirety of the following publications is incorporated herein by reference: Non-Stoichiometric TMDC (MoS₂ and WSe₂) Rapid Energy Prediction and Stable Configuration Search; Towards Invertible 2D Crystal Structure Representation for Efficient Downstream Task Execution (see appendices 1-4).

### SUMMARY OF THE INVENTION

The present invention pertains to a system and method for predicting the physical properties of 2D crystals with defect configurations. The present invention incorporates the use of Siamese Neural Networks (“SNNs”) which are renowned for their ability to learn invariant representation of data. SNNs are able to use the same weights while working in tandem on two different input vectors in order to compute comparable output vectors. SNNs have the advantage of being able to accept inputs of varying sizes, allowing them to adapt to various tasks. Through the use of SNNs, the present invention has a novel ability to incorporate polynomial features which enhances the predictive power. By mapping property space to structural configurations, the present invention facilitates an efficient exploration of solution spaces, opening up possibilities for customized synthesis of new materials.

MoS₂ is a 2D hexagonal crystal with layered structure stacked by alternating layers. Two main types of defects present in MoS₂'s properties are vacancies and substitutions. Vacancies manifest as either molybdenum or sulfur absences, each affecting the lattice differently. Molybdenum vacancies significantly alter the electronic structure and demand high formation energy. In contrast, sulfur vacancies cause moderate lattice disturbances. Substitutional defects include tungsten replacing molybdenum and selenium substituting for sulfur, leading to relatively smaller disruptions due to their analogous outer electron configurations to the original atoms. While MoS₂ is discussed by way of example herein, other materials may be used in place of MoS₂, for example, and not by way of limitation, WSe₂ may be used. WSe₂ has a structure similar to MoS₂, with a plane of tungsten atoms sandwiched between two planes of selenium atoms.

In machine learning (“ML”), “embeddings” are vital data representations that bridge the gap between raw information and effective model learning. These embeddings are essential because they reduce dimensionality, capture meaningful patterns, and enable models to work efficiently with various data types, such as text, images, and categorical variables. They are crucial for enhancing model performance, semantic understanding, similarity measurements, and more. The present invention provides an ability to create invariant embeddings of defect placements in MoS₂, allowing for the capture of critical defect configurations while respecting the crystalline symmetry. In this context, invariance means that embeddings of placements that can be obtained from one another should be closer to each other than the embeddings of placements that cannot be derived from each other.

To evaluate the embedding model, target variables were utilized, including formation energy per site. Formation energy is the energy required to create a specific defect configuration and is defined as follows:

$${E}_{f}^{′} = \frac{E - {E}_{pristine} + {\sum }_{i} {n}_{i} {μ}_{i}}{N}$$

Here, E represents the energy of the configuration with defects, Eₚᵣᵢₛₜᵢₙₑ is the energy of the defect-free configuration, nᵢ is the difference in the quality of the i-th atom in the configuration, μᵢ is the chemical potential of the i-th atom, and N is the number of defects in the supercell. The second target variable is the HOMO-LUMO gap, which represents the difference between the highest occupied molecular orbital (“HOMO”) and the lowest unoccupied molecular orbital (“LUMO”).

Alternative embodiments of the present invention may include alterations to enhance the model's performance. Potential modifications to the preferred embodiment include elimination of the two-stage training process, reduction of the network size, utilization of advanced architectures, application of transfer learning, experimentation with loss functions, incorporation of domain knowledge, and other modifications.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The various embodiments are illustrated by way of example, and not by way of limitation, in the figures of the accompanying drawings. Having thus described the invention in general terms, reference will now be made to the accompanying drawings, which are not necessarily drawn to scale, and wherein:

FIG. 1 is the schematic architecture of an SNN.

FIG. 2 is an illustration of allowed symmetries.

FIG. 3 shows the training method of the present invention.

FIG. 4 illustrates the construction of the final embedding.

FIG. 5 illustrates an energy prediction process for a fixed group.

FIG. 6 is a diagram illustrating the steps of a training network.

FIG. 7 is a diagram illustrating the step-by-step process of the application of a trained model for a downstream task.

### DETAILED DESCRIPTION OF THE PREFERRED EMBODIMENT

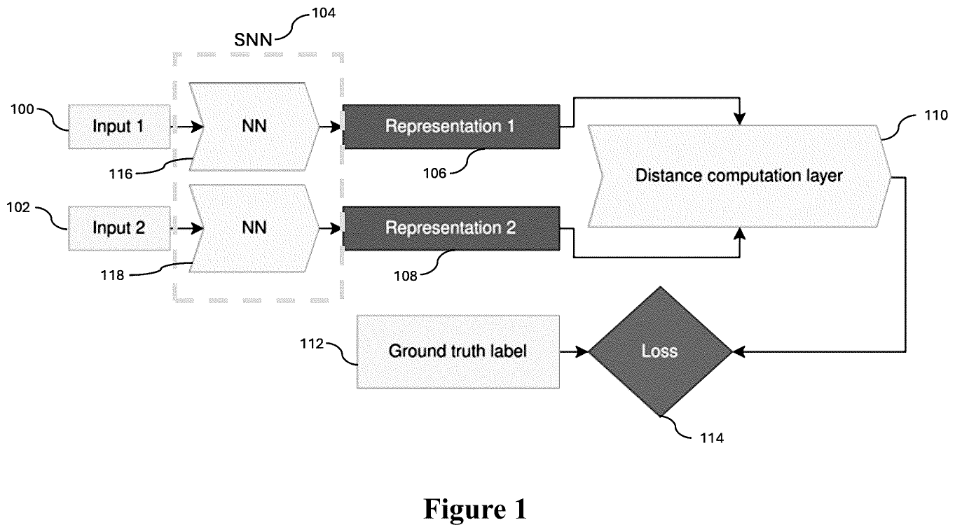

FIG. 1 is the schematic architecture of a SNN. In accordance with the preferred embodiment of the present invention, the SNN 104 (comprising of neural networks (“NNs”) 116 and 118) was trained to generate vector embeddings suitable for representing the differences and similarities between various defect configurations of 2D crystals including but not limited to MoS₂, allowing for the identification of equivalent defect placements and predicting resultant physical properties. Input data 100, 102 is represented as pairs of samples

$$( {x}_{i}^{1} , {x}_{i}^{2} ) ,$$

which are vector representations of the placement. Each sample pair is paired with a yᵢ=21 if

$${x}_{i}^{1} and {x}_{i}^{2}$$

represent placements of the same configuration, and yᵢ=0 otherwise. The final network is composed of several blocks. The initial block 104 is a neural network (“SNN”) applied simultaneously to each input, as illustrated. After the SNN, two embeddings 106, 108 are passed through the distance computation layer 110, transforming them into a single number from the interval [0,1]. Denoting the NN outputs for both inputs as

$$em {b}_{i}^{1} = N N ( {x}_{i}^{1} ) and {emb}_{i}^{2} = N N ( {x}_{i}^{2} ) ,$$

the label is calculated as follows:

$${label}_{i} = 1 - sigmoid ( {‖ {emb}_{i}^{1} - e m {b}_{i}^{2} ‖}_{2} - r )$$

Where r>0 is a fixed hyperparameter. In this scenario, the SNN outputs a probability indicating whether the two placements belong to the same configuration. During training, the goal is to minimize the binary cross-entropy loss function 114 between the SNN output labelᵢ and, 112 the ground truth label yᵢ. Such training procedure guarantees that placements from identical configurations will share the same embedding, with the norm of their distance approaching zero, while placements from distinct configurations will be assigned different embeddings.

In the proposed SNN, the convolutional neural network (“CNN”) plays a crucial role in processing the input data. Specifically, it plays the role of encoder generating the embedding which is invariant to permitted moves. The architecture of the CNN utilized in the present invention includes three convolutional layers and three fully connected layers.

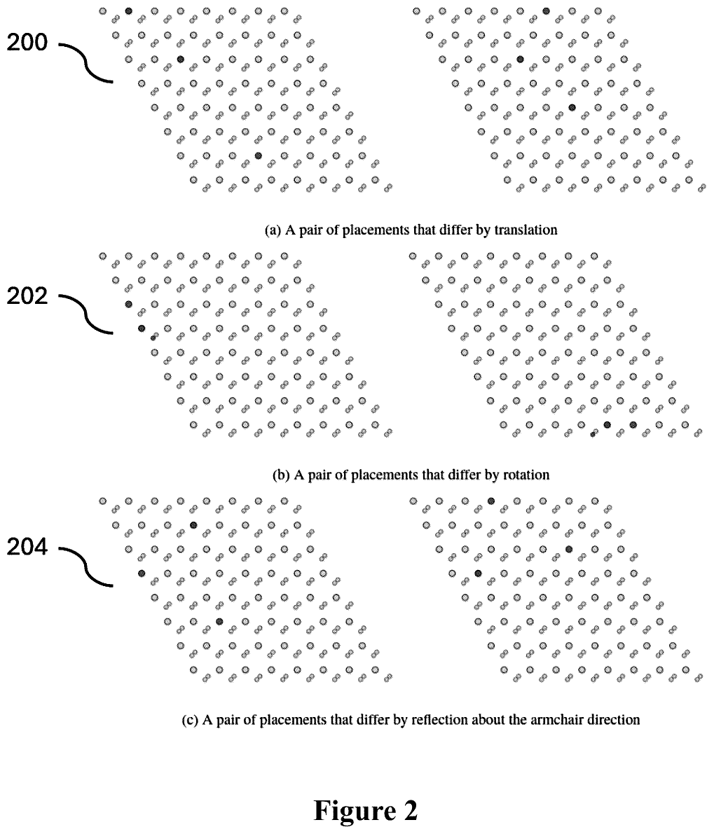

FIG. 2 is an illustration of allowed symmetries. In accordance with the preferred embodiment of the present invention, the configuration of defects can be represented by placing multiple defects on an 8×8 supercell. Even if these placements vary in the coordinates of the defects, they may share the same geometry and, consequently, the same physical properties. FIG. 2 shows several allowed transformations (symmetries) which can be applied to a placement, resulting in a representation of the same configuration. Periodic translations as in 200 (a) occur wherein each defect is moved along a vector (according to the periodicity conditions). Three-fold rotation as in 202 (b) occurs wherein each defect is rotated clockwise around the bottom-left corner of the lattice by 120 degrees. Reflection about the plane of the middle (molybdenum) layer occurs wherein the middle layer remains unchanged, while defects from the outer layers are mirrored to the corresponding position on the opposite layer. Lastly, reflection about the armchair direction as in 204 (c) occurs wherein each defect is mirrored to a symmetrical position within the same layer, across the armchair direction.

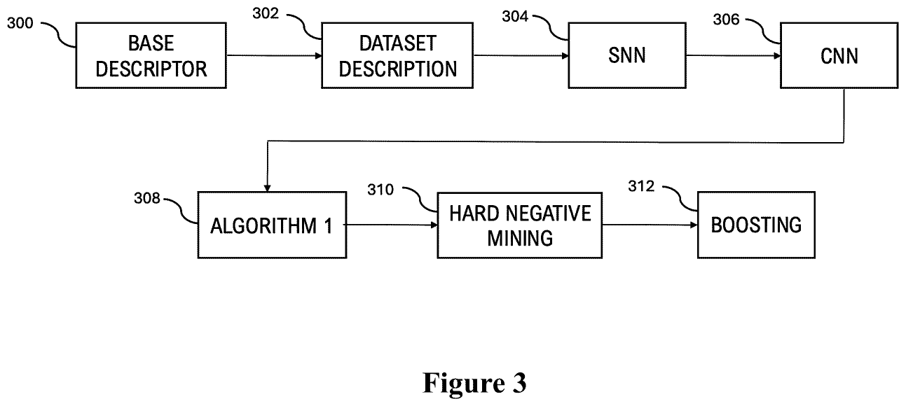

FIG. 3 shows the training method of the present invention. In accordance with the preferred embodiment of the present invention, the method comprises computing a base descriptor 300, describing the dataset 302, training the SNN 304, applying the CNN architecture 306, preparing the dataset for model training using algorithm 1 308, employing hard negatives mining 310, and boosting 312.

The base descriptor 300 of a configuration differentiates placements based on the count of defects for each specific layer and defect type combination, aiming to identify placements that are distinguishable solely by these counts. Once the base descriptor is computed for each combination of layer and defect types, the dataset is described 302. In one embodiment, the dataset may be described as comprising all possible configurations with exactly three defects. Each configuration can be manifested through multiple placements. The SNN 304 is then utilized to derive a descriptor for each placement, and the CNN 306 is employed to process the input data. Using a transformation algorithm that applies reflections, rotations, and translations to a randomly sampled placement from a given configuration (Algorithm 1 308), the dataset is prepared for model training. To guard against receiving a true negative rate less than 1 due to false positives, hard negative mining 310 is employed. Hard negatives are pairs of configurations with overlapping embeddings. Following the hard negative mining 310, a process referred to as boosting 312 is utilized to target and amplify the training emphasis on challenging pairs, improving the model's ability to correctly classify them and thereby enhancing overall performance. The method of the present invention provides a novel SNN approach capable of creating invariant embeddings for 2D crystal defect configurations.

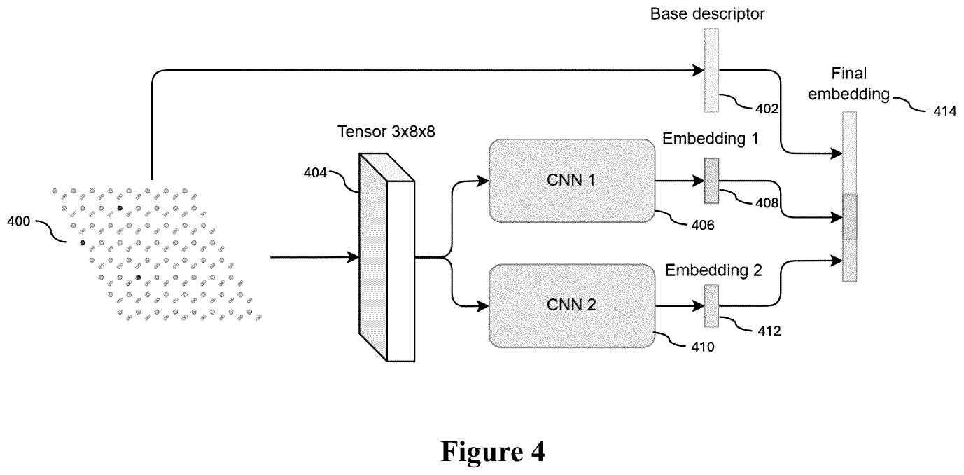

FIG. 4 illustrates the construction of the final embedding 414. In accordance with the preferred embodiment 400 of the present invention, the final embedding is constructed in three parts. A first part of the final embedding is constructed via the base descriptor 402 of a configuration 400. As described in FIG. 3, the base descriptor 402 differentiates placements based on the count of defects for each specific layer and defect type combination. Embedding 1 (408) and embedding 2 (412) of the final embedding 414 comes from the tensor 404 of the configuration. Every placement is treated as an image comprising three layers, represented by a tensor with the shape [3, 8, 8]. A first 406 and second 410 CNN are employed on the tensor 404, providing the second 408 and third 412 parts (embeddings 1 and 2) of the final embedding 414, respectively. Other construction routes for the final embedding 414 are contemplated herein.

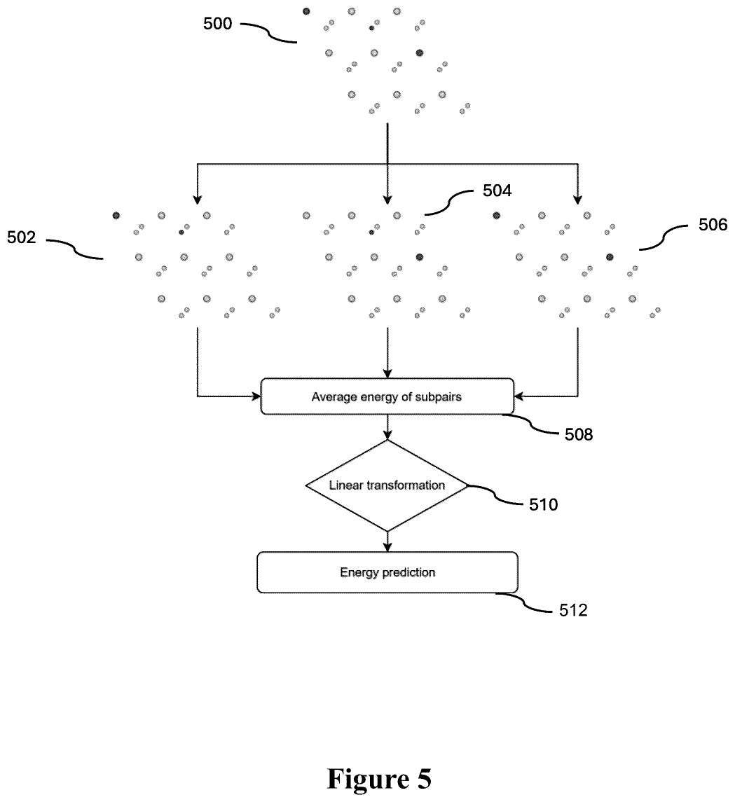

FIG. 5 illustrates an energy prediction process for a fixed group. In accordance with the preferred embodiment of the present invention, groups may be defined based on atom counts. A linear model may be developed to link a configuration's energy 500 with the average energy of its subpairs 502, 504, 506 within the specific group. A group is a set of configurations of defects on a particular material defined by numbers V_{M}, V_{X}, S_{M}, and S_{X}. Configuration A is said to be a subpair of configuration B if A contains only two defects and can be obtained from B by omitting all defects except for some two. The average subpairs energy 508 of configuration A is the average energy of all subpairs of A, denoted as ε_{avg}(A).

The energy of a configuration can be estimated by the average energy of all its subpairs 508. A linear regression 510 may be employed to establish a dependency between the energy of a configuration and the average energy of all its subpairs:

$${\hat{E}}_{g r o u p} ( A ) = w \cdot {ε}_{a v g} ( A ) + b$$

Where A is the configuration for which we aim to predict the energy, Ê_{group} is the approximation for energy inside the group of configuration A, and w and b are the weight and bias of the linear dependence, respectively. The parameters w and b are group-specific, meaning that they are constant for one group but may vary across different groups. The energy prediction 512 pipeline is illustrated in FIG. 5. The model may then be generalized for various defect groups and further expanded to accommodate any number of defects for a specific material. The linear model 510 can be generalized to all groups with a specific number of defects:

$${\hat{E}}_{n} ( A ) = w \cdot {ε}_{a v g} ( A ) + {V}_{M} \cdot {θ}_{{V}_{M}} + {V}_{X} \cdot {θ}_{{V}_{X}} + {S}_{M} \cdot {θ}_{{S}_{M}} + {S}_{X} \cdot {θ}_{{S}_{X}}$$

Here, A represents the configuration with n defects, Êₙ(A) is the energy approximation for configurations with n defects. The symbols V_{M}, V_{X}, S_{M}, and S_{X} denote the counts of vacancies and substitutions on the metal and chalcogen layers, respectively, within configuration A. The parameters w, θ_{V}_{_{M}}, θ_{V}_{_{X}}, θ_{S}_{_{M}}, θ_{S}_{_{X}} are cardinality-specific and vary with the number of defects. These parameters must be determined separately for each defect count. Furthermore, the above model can be further generalized to accommodate an arbitrary number of defects by modifying only the coefficients surrounding the entire sum as follows:

$${\hat{E}}_{material} ( A ) = {w}_{n} \cdot {ε}_{a v g} ( A ) + {θ}_{n} ( {V}_{M} \cdot {θ}_{{V}_{M}} + {V}_{X} \cdot {θ}_{{V}_{X}} + {S}_{M} \cdot {θ}_{{S}_{M}} + {S}_{X} \cdot {θ}_{{S}_{X}} ) + {b}_{n}$$

In this formulation, Êₘₐₜₑᵣᵢₐₗ is an approximation of the energy independent of the number of defects, where θ_{V}_{_{M}}, θ_{V}_{_{X}}, θ_{S}_{_{M}}, θ_{S}_{_{X}} are parameters not dependent on the number of defects. The parameters wₙ, θₙ, bₙ are specific to each cardinality and need to be determined for each number of defects.

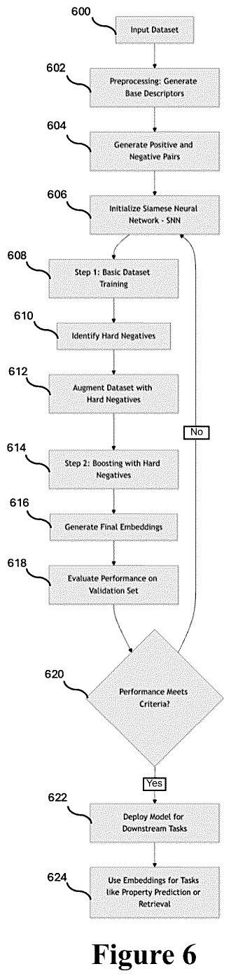

FIG. 6 is a diagram illustrating the steps of a training network. First, the training network undergoes Input Dataset 600 in which the dataset contains defect configurations and placements, and the dataset may be inputted. Then, in 602 Preprocessing: Generate Base Descriptors, invariant base descriptors are generated by counting defects and applying symmetry transformations. Then, in 604 Generates Positive and Negative Pairs, pair generation occurs in which pairs of placements are created and labeled as positive or negative. Then, in 606 Initializes Siamese Neural Network (SNN), SNN initialization occurs in which a Siamese Neural Network is set up to process pairs of embeddings. 608 Step 1: Basic Dataset Training is then underwent wherein training occurs in which training on the dataset with a basic loss function occurs. After, in 610 Identify Hard Negatives, identification occurs in which cases where embeddings are misclassified (i.e. false positives) are identified. Then, in 612 Augment Dataset with Hard Negatives, augmentation occurs in which hard negatives are added to the training dataset. 614 Step 2: Boosting with Hard Negatives is then undergone wherein a second round of training occurs in which the network is retrained with the augmented dataset. Then, in 616 Generates Final Embeddings, generation embedding occurs in which final invariant embeddings are produced. Then, in 618 Evaluates Performance on Validation Set, performance evaluation occurs wherein the embeddings are validated for their effectiveness. After, it is determined whether the Performance Meets the Criteria 620 with one of two outcomes possible. If the performance meets the criteria, then the result is 622 Deploy Model for Downstream Tasks in which deployment occurs wherein the trained model is used for practical downstream tasks. With the criteria met, then 624 Uses Embeddings for Tasks like Property Prediction or Retrieval occurs. However, if the performance does not meet the criteria, then the result requires the model to undergo refining. The system thus returns to 606 Initializing SNN, and, once the model is refined, SNN initialization occurs, and the system proceeds to 608 Step 1 as previously determined.

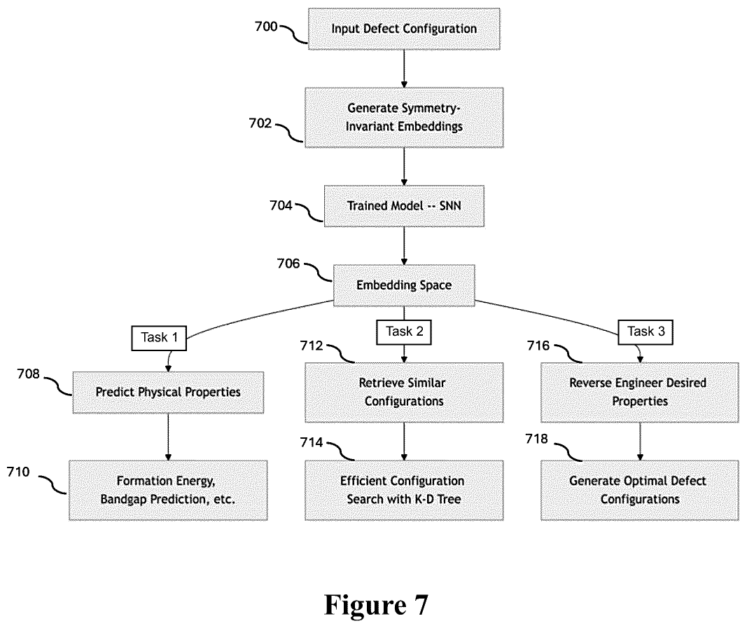

FIG. 7 is a diagram illustrating the step-by-step process of the application of a trained model for a downstream task. First, in 700 Input Defect Configuration, the raw defect configuration (e.g. defect placements in a lattice) is provided. Then, in 702 Generates Symmetry-Invariant Embeddings, the input is processed through the trained model to obtain embeddings invariant to lattice symmetries. The result of this generation is 704 a Trained Model-SNN. Then, in 706 Embedding Space, the embeddings represent the defect configuration in a compact, high-dimensional space. Embedding Space results in three different Tasks: Task 1, Task 2, and Task 3, each with its own result. Task 1, or 708 Predict Physical Properties, involves using embeddings for property prediction tasks like formation energy or bandgap. The result is 710 Formation Energy, Bandgap Prediction, etc. Task 2, or Retrieve Similar Configurations 712, involves performing efficient similarity searches using a K-D tree or similar methods. The result is 714 Efficient Configuration Search with K-D Tree. Task 3, or 716 Reverse Engineer Desired Properties, involves mapping desired physical property ranges back to defect configurations via generative techniques. The result is 718 Generate Optimal Defect Configurations. Further results of the Tasks can be accurate predictions of physical properties, fast retrieval of similar configurations, and generation of defect configurations tailored to specific material properties.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A system for predicting physical properties of two-dimensional crystals with defect configurations, comprising:
at least one machine learning model used to generate invariant embeddings of defect configurations in two-dimensional crystal lattices;
said at least one machine learning model applying a training methodology to defect recognition;
a Siamese Neural Network (SNN) trained on labeled pairs of defect placements, wherein said labeled pairs of defect placements comprise positive pairs representing identical configurations derived through symmetry operations and negative pairs representing distinct configurations with similar base descriptors; and
a distance-based loss function configured to optimize said embeddings for classification accuracy;
a base descriptor computationally generated for defect configurations in two-dimensional crystal lattices;
a convolutional neural network architecture utilizing circular padding to ensure consistent feature representation during periodic translations of input data, enhancing invariance to defect placements;
a predictor of physical properties of two-dimensional materials;
a retrieval of lattice configurations based on said embeddings;
a generalized approach for invariant embedding generation that applies to various two-dimensional materials and accommodates different defect densities; and
a designing of two-dimensional crystal structures.

2. The system of claim 1, wherein said machine learning model includes:
utilizing said SNN to create embeddings invariant to symmetry operations such as translation, rotation, and reflection specific to a lattice structure; and
employing a contrastive learning framework to ensure embeddings of equivalent configurations are close in an embedding space, while non-equivalent configurations are distant.

3. The system of claim 1, wherein said base descriptor computationally generated includes:
counting defect occurrences across lattice layers and types; and
applying symmetry-based transformations to ensure descriptor invariance under reflection or rotation.

4. The system of claim 1, wherein said training methodology includes:
initial training with a balanced dataset of positive and negative pairs;
identifying hard negatives and incorporating them into a training dataset for enhanced discrimination.

5. The system of claim 1, wherein said predictor of physical properties of two-dimensional materials includes:
mapping invariant embeddings to target physical properties, including formation energy and electronic bandgap; and
employing a multi-layer perceptron (MLP) for downstream tasks, trained on embeddings augmented with polynomial features for enhanced predictive accuracy.

6. The system of claim 1, wherein said retrieval of lattice configurations based on embeddings includes:
a K-D tree utilized for efficient nearest-neighbor searches in an embedding space; and
enabling rapid identification of configurations with desired properties.

7. The system of claim 1, wherein said generalized approach for invariant embedding generation that applies to various two-dimensional materials and accommodates different defect densities further by:
standardizing input representations; and
preserving invariance under symmetry operations regardless of defect count.

8. The system of claim 1, wherein said designing of two-dimensional crystal structures includes:
mapping desired physical property ranges to specific defect configurations using a learned embedding space; and
employing generative models trained on embeddings to propose new configurations.

9. A method for generating invariant embeddings of defect configurations in two-dimensional crystal lattices using a machine learning model, the method comprising:
utilizing a neural network to create embeddings invariant to symmetry operations specific to a lattice structure;
employing a contrastive learning framework to ensure embeddings of equivalent configurations are close in an embedding space, while non-equivalent configurations are distant;
generating invariant embeddings of defect configurations in two-dimensional crystal lattices using a machine learning model;
training a Siamese Neural Network on labeled pairs of defect placements;
optimizing said embeddings for classification accuracy through a distance-based loss function;
computationally generating a base descriptor for defect configurations in two-dimensional crystal lattices;
training for machine learning models applied to defect recognition;
utilizing circular padding within a convolutional neural network architecture to ensure consistent feature representation during periodic translations of input data, enhancing invariance to defect placements;
predicting physical properties of two-dimensional materials;
retrieving lattice configurations based on embeddings;
applying invariant embedding generation to various two-dimensional materials and accommodates different defect densities; and
designing two-dimensional crystal structures for generation.

10. The method of claim 9, wherein said symmetry operations include translation, rotation, and reflection.

11. The method of claim 10, wherein said machine learning model includes:
utilizing a neural network to create embeddings invariant to symmetry operations such as translation, rotation, and reflection specific to a lattice structure; and
employing a contrastive learning framework to ensure embeddings of equivalent configurations are close in an embedding space, while non-equivalent configurations are distant.

12. The method of claim 10, wherein said Siamese Neural Network includes:
positive pairs representing identical configurations derived through symmetry operations; and
negative pairs representing distinct configurations with similar base descriptors.

13. The method of claim 10, wherein said base descriptor computationally generated includes:
counting defect occurrences across lattice layers and types; and
applying symmetry-based transformations to ensure descriptor invariance under reflection or rotation.

14. The method of claim 10, wherein a training methodology includes:
initial training with a balanced dataset of positive and negative pairs; and
identifying hard negatives and incorporating them into a training dataset for enhanced discrimination.

15. The method of claim 10, wherein a predictor of physical properties of two-dimensional materials includes:
mapping invariant embeddings to target physical properties, including formation energy and electronic bandgap; and
employing a multi-layer perceptron (MLP) for downstream tasks, trained on embeddings augmented with polynomial features for enhanced predictive accuracy.

16. The method of claim 10, wherein a retrieval of lattice configurations based on embeddings includes:
a K-D tree utilized for efficient nearest-neighbor searches in an embedding space; and
enabling rapid identification of configurations with desired properties.

17. The method of claim 10, wherein a generalized approach for invariant embedding generation that applies to various two-dimensional materials and accommodates different defect densities further by:
standardizing input representations; and
preserving invariance under symmetry operations regardless of defect count.

18. The method of claim 10, wherein a designing of two-dimensional crystal structures includes:
mapping desired physical property ranges to specific defect configurations using a learned embedding space; and
employing generative models trained on embeddings to propose new configurations.

19. A system for predicting physical properties of two-dimensional crystals with defect configurations, comprising:
at least one machine learning model used to generate invariant embeddings of defect configurations in two-dimensional crystal lattices;
said at least one machine learning model applying a training methodology to defect recognition;
a Siamese Neural Network trained on labeled pairs of defect placements;
a distance-based loss function to optimize the embeddings for classification accuracy;
a base descriptor computationally generated for defect configurations in two-dimensional crystal lattices;
a convolutional neural network architecture utilizing circular padding to ensure consistent feature representation during periodic translations of input data, enhancing invariance to defect placements;
wherein said convolutional neural network architecture includes three convolutional layers and three fully connected layers;
a predictor of physical properties of two-dimensional materials;
a retrieval of lattice configurations based on embeddings;
a generalized approach for invariant embedding generation that applies to various two-dimensional materials and accommodates different defect densities;
wherein application and accommodation includes standardizing input representations and preserving invariance under symmetry operations regardless of defect count;
a designing of two-dimensional crystal structures; and
an ability to enhance a classification of defect configurations by concatenating a base descriptor of defects, embeddings generated from multiple stages of neural network processing; and distinct components derived from hierarchical training.

20. The system of claim 19, wherein a final embedding is constructed in three parts, comprising:
a first part constructed via the base descriptor of a configuration; and
a second part and a third part deriving from a tensor of the configuration.
